(19) United States
(12) Patent Application Publication
(10) Pub. No.: US 2014/0218690 A1
(43) Pub. Date: Aug. 7, 2014

(54) METHOD AND DEVICE FOR ASCERTAINING AND MAPPING DATA REPRESENTING THE OPTICAL IMAGING PROPERTIES OF AN EYE

(71) Applicants: Sven Heinrich, Gundelfingen (DE); Michael Bach, Gundelfingen (DE)

(72) Inventors: Sven Heinrich, Gundelfingen (DE); Michael Bach, Gundelfingen (DE)

(21) Appl. No.: 14/241,090

(22) PCT Filed: Aug. 17, 2012
(86) PCT No.: PCT/EP2012/003521

§ 371 Date: Feb. 26, 2014

(30) Foreign Application Priority Data
Aug. 26, 2011 (DE) 10 2011 111705.2

(51) Int. Cl. A61B 3/00; A61B 3/09; A61B 3/032

(52) U.S. Cl. CPC A61B 3/0033; A61B 3/032; A61B 3/09
USPC 351/223

(57) ABSTRACT

A method and a device for ascertaining and mapping data representing the optical imaging properties of an eye of a person, in which n different test images are displayed individually in succession on an electronically controllable monitor unit, are seen by the person from a settable viewing angle and at a settable distance, and are identified by means of an input comprising: a) ascertaining a characteristic viewing angle value at a given distance, at which a first probability for correct identification of the test images is achieved, b) ascertaining a characteristic distance value while maintaining the ascertained characteristic viewing angle value, at which a second probability for correct identification is achieved which is greater than the first probability, c) repeating steps a) and b).

### CROSS REFERENCE TO RELATED APPLICATION

Reference is made to German Patent Application Serial No. DE 10 2011 111 705.2, filed on Aug. 26, 2011, which application is incorporated herein by reference in its entirety.

### BACKGROUND OF THE INVENTION

The invention relates to a method for ascertaining and mapping data representing the optical imaging properties of an eye of a person, in which n different test images are displayed individually in succession, preferably in a pseudo-random repeated sequence, on an electronically controllable monitor unit, with the test images being viewed by the person from a settable viewing angle, at a settable distance and being identified by an input.

### DESCRIPTION OF THE PRIOR ART

The optical imaging properties of the eye determine a person's vision, both in terms of visual acuity, that is to the ability to see sharp images or objects which are perceptible through contrasts, and also in terms of optical resolution. Optical resolution characterizes the distinguishability of fine structures, that is to say the smallest distance still perceptible between objects in dot or line form. Accommodation ability, that is to say the dynamic adaptation of the refractive power of the eye in order to sharply image on the retinal plane objects arranged at different distances, is closely linked to the optical imaging properties of the eye. The accommodation ability and here in particular what is known as the near accommodation, that is to say the ability to sharply see, due to adaptation of the refractive force of the eye lens, an object arranged in the vicinity, is gradually lost with increasing age.

To measure and therefore assess a person's vision, either separate visual acuity tests are carried out at different distances, as is known per se, or an object is moved from afar towards the eye to be examined until the person states that they are no longer able to see the object sharply. The first-mentioned method requires eye charts, on which optotypes to be identified by the person are displayed in different forms and sizes. These are to be calibrated accordingly in accordance with the distances at which the eye charts are positioned relative to the person. The last-mentioned method is usually carried out with the aid of optometers, in which an object is positioned at different distances from the eye.

Modern vision test units however make the use of eye charts to be positioned at different distances superfluous, particularly as the optotypes to be recognised by a person can be displayed in different forms and sizes on an electronically controllable monitor and can be displayed via suitable imaging optics with different accommodation requirements for the person. Reference can be made to DE 195 01 415 A1 and EP 1 585 438 B1 for representative examples of vision test units of this type.

Besides conventional methods for establishing a person's vision capability, which are dependent on the cooperation of the person, wherein the person has to identify each of the viewed optotypes or test symbols, test methods that can be carried out fully autonomously, that is to say without cooperation of the person, are also known. Methods of this type use the evaluation of wavefront information of the eye, which is evaluated with the aid of a retina image quality function in order to ascertain a sharpness measure. To this end, a representative compilation of suitable quotations from the literature can be inferred from DE 10 2005 054 691 A1. Such methods can indeed measure the purely optical properties of the eyes, but do not ascertain a person's vision capability. Even if the “eye system” is perfect, perceptual disturbances may still occur, caused by faulty photoreceptors in the retina or by damage in the visual centre in the cerebral cortex, such that the person in question sees poorly or does not see anything.

### SUMMARY OF THE INVENTION

The invention is a method and a device for ascertaining and mapping data representing the optical imaging properties of an eye, in which the accommodation ability of an eye of a person in the near range is to be reliably documented with high reliability and reproducibility. To this end, the examination time is to be as short as possible and the need for cost-relevant resources is to be reduced. The method, where possible, is to be executable without examination staff, such that merely the person to be examined communicates with the device via a technical input as the test is being carried out.

The method according to the invention for ascertaining and mapping data representing the optical imaging properties of an eye of a person uses a vision test unit that is known per se and that has an electronically controllable monitor unit, on which n different test images are illustrated individually in succession. The n test images differing in terms of form and size that can be displayed in a variable manner on the monitor unit can also be imaged onto the eye to be examined of the person at distances from the person that can be set in a variable manner. Via an input means, which enables manual or verbal communication with the person, a piece of test information identifying the test image viewed by the person is used as a basis for the implementation of the vision test system according to the invention.

The method according to the invention for ascertaining and mapping data representing the optical imaging properties of an eye of a person is based on determining and mapping the perceptual capability of the eye by determining the relative frequency of the correct identification of test images in the near range of the eye in accordance with the size and distance of the test images. The limit between correct and incorrect perception of test images at what is known as the optical near point therefore is not precise. Rather, there is a wider or narrower transition region, of which the size and manifestation is specific to each person and characterises the ability and inability of a person to correctly identify test images at the near point or in the near point region in accordance with distance and viewing angle. This transition range varies in a manner specific to each individual person and changes due to ageing over the course of age-related long-sightedness, which generally occurs gradually and is associated with the loss of the ability of the eye to focus on near objects by means of accommodation.

### BRIEF DESCRIPTION OF THE DRAWINGS

FIG. 1 is a graph of the distance s along the abscissa at which a test image is spaced from eye to be examined relative to the angle α along the ordinate at which the person views a test image spaced at the distance.

FIG. 2 is a graph of the distance s at which a test image is spaced from the eye being tested relative to the viewing angle α at which bracketing of the transition region Ü is produced.

FIG. 3 illustrates the use of the method of the invention with three Landolt rings.

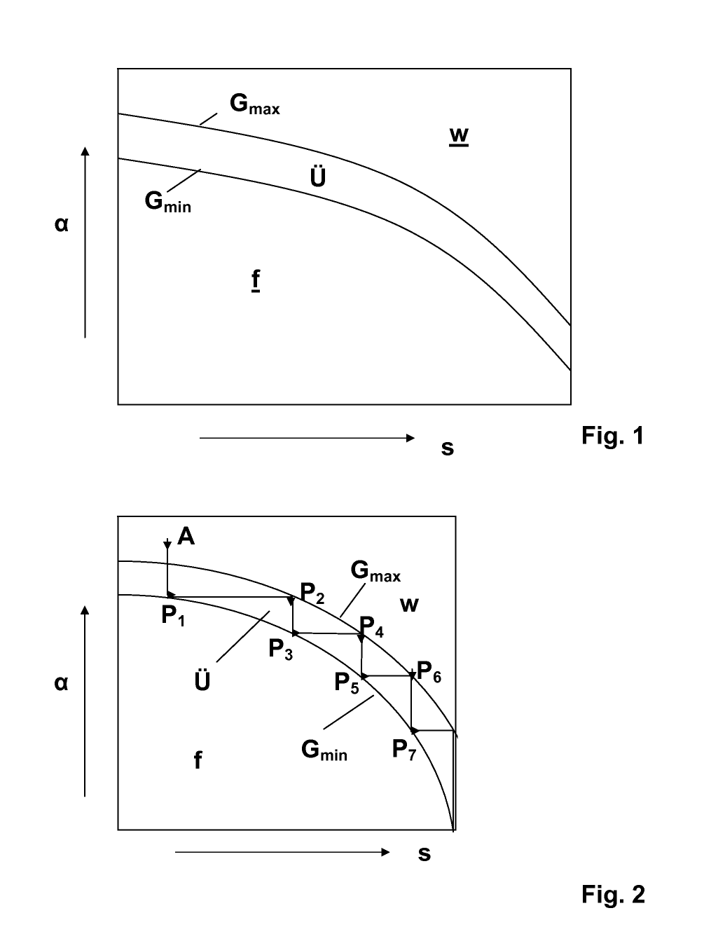

This correlation is illustrated in FIG. 1 for improved understanding. FIG. 1 shows a diagram with an abscissa, along which the distance s is illustrated at which a test image is distanced from the eye to be examined of a person. Along the ordinate, the viewing angle α is plotted from which the eye of a person views a test image shown at a specific distance s on an electronically controllable monitor unit. The function courses Gₘᵢₙ, Gₘₐₓ shown in the diagram indicate the probabilities with which the person is able to correctly identify the test images brought into view in accordance with the distance s and viewing angle α. In this case, the function course Gₘᵢₙ indicates those combinations of viewing angle α and distance s with which the person is still able, only purely statistically, to correctly identify the test images to be identified. Gₘᵢₙ in this case represents a rate probability which, with a presentation of a total of n=8 different test images, is ⅛=12.5%.

Of course, it is possible to select Gₘᵢₙ individually, as will also be explained further below. All combinations of viewing angle α and distance s that are arranged in the diagram below the function course are thus test configurations in which the person cannot correctly identify the test images, that is to say the test results are incorrect f. By contrast, the function course Gₘₐₓ represents those combinations of viewing angle α and distance s with which the person is able to correctly identify the viewed test images with sufficient probability. It is not absolutely necessary to select Gₘₐₓ=100%. Typically, values in the range between 80 and below 100% can be selected. All combinations of viewing angle α and distance s arranged above the function course Gₘₐₓ therefore represent test configurations in which the person is able to correctly identify the test images. This range is denoted by reference sign w.

### DETAILED DESCRIPTION OF THE INVENTION

The objective is to determine the transition region U of a person, limited on either side in FIG. 1 by the lines Gₘᵢₙ and Gₘₐₓ, and to map the transition region u in the presentation illustrated in FIG. 1 in the form of a two-dimensional psychometric function characterizing a person's vision.

In order to implement this economically and in a manner that is as time-saving as possible, it is sufficient to examine the vision or the optical imaging properties of the eye of a person merely on the basis of those measurement parameters, that is to say viewing angle and distance, that lie or are expected to lie within and/or around the transition region Ü, particularly since the course of the transition region Ü in individual people is not clear a priori.

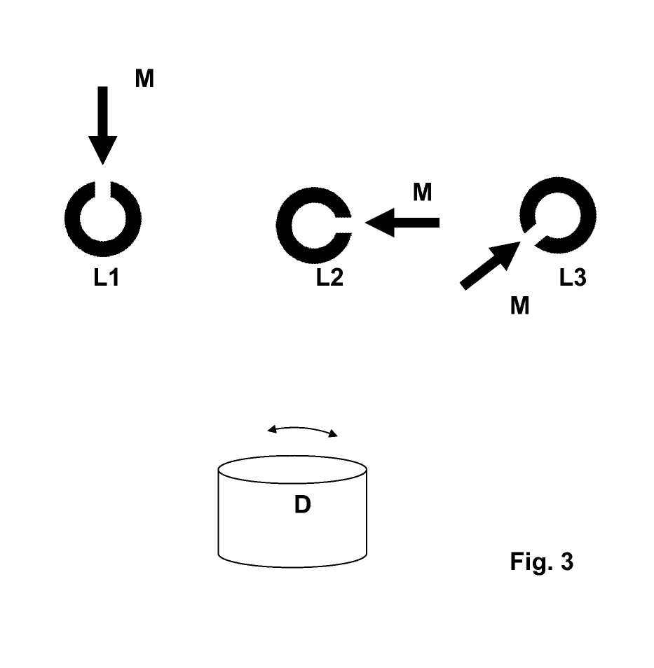

To measure this transition region, it is possible in principle to select an arbitrarily selected initial combination of viewing angle and distance, with which a test image selected from n different test images is brought into a person's view. However, an initial combination with which the person sharply recognises the test image and is therefore able to identify the test image reliably is preferably selected. An initial combination of this type is indicated in FIG. 2 as the starting point A, which is characterised by a large viewing angle α and a short distance s. Proceeding from the starting point A, various test images are shown to the person in gradual sequence, wherein the viewing angle α is gradually reduced at each unchanged distance s. In this case, the person finds it more difficult however with increasing reduction of the viewing angle α to correctly identify the respective test image viewed. The number of incorrectly identified test images therefore rises. Working on the basis of an adaptive psychometric threshold determination, which will be explained in greater detail further below, the iterative viewing angle reduction with constant distance is implemented accordingly and aborted as soon as the person for example starts merely to guess the test images presented to him. This is the case when a function point P₁ of the function course Gₘᵢₙ is reached. Proceeding from this function point P₁, the viewing angle α is subsequently kept constant, however the distance at which individual test images are presented separately to the person is gradually enlarged. The person thus finds it easier with increasing distance to correctly identify the individual test images. The approaching of a function point P₂ of the function course Gₘₐₓ, which defines a parameter combination of viewing angle and distance with which the person is able to correctly identify the respective viewed test image with a predefinable acceptable probability of recognition, is in turn implemented with the aid of an adaptive psychometric threshold determination to be explained in greater detail further below. Both aforementioned threshold determinations are carried out repeatedly in alternation in order to determine the further function points P₃ to P₇ shown schematically in FIG. 2, whereby a step-like bracketing of the transition region Ü is produced.

The above-explained method for ascertaining and mapping data representing the optical imaging properties of an eye of a person is thus based on the following:

In a first method step, starting from a freely selectable distance, which corresponds to the distance between the test image presented on an electronically controllable monitor unit and the eye of the person, and keeping this distance constant, a characteristic viewing angle value at which the person correctly identifies individual test images with a predefinable first probability is ascertained. The predefinable first probability corresponds in the case of the illustration in FIG. 2 to Gₘᵢₙ. Keeping the obtained characteristic viewing angle value constant, a characteristic distance value at which the person can correctly identify the test images with a second probability that is greater than the above first-mentioned probability and corresponds to the probability Gₘₐₓ explained in FIG. 2 is then determined in a second method step. Both of the aforementioned steps are repeated, wherein the characteristic distance value ascertained in the second step is used in the first step as a given distance until the distance at which the person views the test image ultimately corresponds to a final distance. The characteristic viewing angle values and characteristic distance values ascertained here are mapped or displayed in accordance with viewing angle and distance in a two-dimensional diagram for illustration of the transition region. The transition region describes the probability distribution in accordance with viewing angle and distance with which a person is able to correctly recognise test images in the near range around the near point.

The method according to the invention uses a vision test unit known per se, with which the test images to be viewed by a person can be displayed on an electronically controllable monitor unit. The monitor unit is arranged such that it can be moved along a linear unit bi-directionally relative to the eye of the person or to a chin rest fitted on the vision test unit. With the aid of a vision test unit of this type, on the one hand the distance between the eye and test image and on the other hand the viewing angle are possible by variable size display of each individual test image on the monitor unit. Alternatively to a monitor unit arranged in a linearly movable manner, the distance at which a person perceives an optotype may also vary with the aid of a suitable imaging optics. To this end, the imaging optics is arranged between the chin rest and the fixedly arranged monitor unit, and either optical lenses having a positive optical refractive value reducing the accommodation requirement or optical lenses having a negative optical refractive value increasing the accommodation requirement can be introduced into the imaging optics in order to vary the accommodation requirements.

A device suitable for carrying out the method according to the invention additionally comprises a control unit, which selects individual test images that can be displayed on the monitor unit. Furthermore, the control arrangement scales the test image size on the basis of a selection rule, whereby the viewing angle from which the person views a test image can be set individually. Lastly, the control arrangement controls the linear unit on the basis of a further selection rule for setting a specific distance between the monitor unit and the chin rest or the eye of the person.

Furthermore, an input is provided, via which the person generates a piece of test information identifying the test image. The input means is preferably formed in such a way that the person does not have to change his gaze from the monitor unit during the input. For this purpose verbally or manually operable inputs are provided, in particular inputs that can be operated intuitively.

Lastly, an evaluation unit is provided that, on the basis of at least the first selection rule, ascertains a characteristic viewing angle value, at least at one given distance by systematic, iterative reduction of the viewing angle, at which a first probability for correct identification of the test images is achieved. Furthermore, with the aid of the evaluation unit, a characteristic distance value is ascertained on the basis of at least the second selection rule by systematically enlarging the distance at which a second probability for correct identification of the test image can be achieved, which is greater however than the first probability, while maintaining the at least one ascertained characteristic viewing angle value. With the aid of a display unit that is likewise provided, for example a second monitor unit or a conventional paper printout, all ascertained characteristic viewing angle and distance values can be displayed and/or mapped in accordance with viewing angle and distance.

It is of course possible, by providing a memory unit, to store at least the characteristic viewing angle and distance values for subsequent processing or archiving.

To explain the above-mentioned adaptive psychometric threshold determination, two preferred method variants will be described hereinafter, with which characteristic viewing angle and distance values can be established and can be mapped within the scope of a two-dimensional display.

To carry out the testing of the optical imaging properties of the eye of a person, standardised optotypes are preferably used as test images, for example what are known as Landolt rings, which consist of n=8 different forms of appearance. Of course, other optotypes can also be used to carry out the method according to the invention, such that the reference hereinafter to Landolt rings is not intended to limit the general inventive concept.

In a first step, a test image is selected from the n=8 different Landolt rings and this is moved from the eye of the person to a first predefined distance s and is brought into view from a first viewing angle α with the aid of a vision test unit. As already mentioned before, the initial situation should be selected in such a way that the starting distance between the test image and eye is selected to be as small as possible and the test image size and the viewing angle associated therewith are selected to be as large as possible, such that the person sees the first test image as sharply as possible and therefore reliably indentifies it. For identification, the person activates a suitable input, preferably without changing their view from the electronically controllable monitor unit, in order to generate data pertaining to test information that is subsequently classified as incorrect or correct. In the case of a correct recognition of the test image, a further test image is displayed for identification in a next step at the same distance, but from a reduced viewing angle of the person. The aforementioned gradual reduction of the viewing angle occurs repeatedly with correct identification, wherein the viewing angle is in each case reduced gradually by a constant measure. If, by contrast, the person incorrectly identifies the test image displayed at the constant distance, the viewing angle in the next step is thus not reduced as before, but increased by a smaller measure compared to the above-described reduction measure. If the next recognition step likewise leads to an incorrect identification of the differently selected test image, the viewing angle is thus displayed again in a manner enlarged by the corresponding measure, whilst keeping the distance constant. On the basis of the above-explained gradual procedure, the viewing angle converges with increasing sequence of steps towards a characteristic viewing angle value, at which it is assumed that the probability for correct identification corresponds to the rate probability, which, with n=8 different Landolt rings, is purely mathematically 12.5%. When a characteristic viewing angle value of this type is reached, the further sequence of steps in this regard can be aborted.

In accordance with the invention, once the characteristic viewing angle value has been determined at the predefined distance s, a changed further sequence of steps is then implemented, in which the viewing angle corresponding to the ascertained characteristic viewing angle is kept constant and the distance is enlarged gradually. The distance is always enlarged in those cases in which the person incorrectly identifies the test image. If the person correctly recognizes the displayed test image, the test image is thus presented in the next step at a slightly shorter distance. In contrast to the prior procedure, the reduction measure by which a distance reduction is undertaken is now selected so as to be smaller than the enlargement measure by which the distance is further distanced gradually from the eye of the person in the event of a corresponding incorrect identification of a test image. In this case too, the distance values, which can be set in a variable manner, converge towards a characteristic distance value, which cannot change or cannot noticeably change in spite of further repetition of steps in this respect. The characteristic distance value obtained in this way at the ascertained characteristic viewing angle value kept constant indicates the distance between the test image and eye at which the person can correctly identify the test images with a sufficiently high probability. The aforementioned two sequences of steps, specifically changing the viewing angle with constant distance and changing the distance with constant viewing angle, can in principle be repeated as often as desired until a final distance is achieved that corresponds for example to the smallest achievable accommodation distance of the person, such that, with a further enlargement of the distance with constant viewing angle, there is no improvement of the identification ability of the test person.

A second alternative procedure for determining and mapping data representing the optical imaging properties of an eye of a person, the data is in the form of characteristic viewing angle and distance values, provides a statistical evaluation on each individual measurement configuration. Starting with the initial configuration at point A according to FIG. 2, the measurement is repeated a number of times, that is to say test images for identification are presented a number of times in succession to the person in order to thus ascertain the relative frequency for correct recognition or identification of the test image at point A. This is carried out for each individual measurement configuration. It is therefore unnecessary to reduce the viewing angle gradually, as described above, and to enlarge it again as necessary, rather the viewing angle is reduced gradually by a constant measure, wherein the statistical frequency with which the person is able to correctly identify the different test images is calculated at each individual viewing angle. If a viewing angle is reached at which the ascertained relative frequency corresponds to a lower limit value Gₘᵢₙ, this thus corresponds to the characteristic viewing angle. The characteristic distance that is then present when the relative frequency corresponds to an upper limit value Gₘₐₓ is ascertained similarly. With corresponding repetition, the points P₁ to P₇ illustrated in FIG. 2, which, as characteristic points, define a step function and thus characterise the transition region Ü, are thus obtained.

A possibility for forming an input which is to be operated intuitively and which is suitable for use by a person for the identification in particular of Landolt rings without the person having to change their view from the electronically controllable monitor unit is illustrated schematically in FIG. 3. The three illustrated Landolt rings L1, L2, L3, from a total of eight different Landolt rings, each have a gap that can be located in one of eight possible positions. A mark M that can be imaged beside the respective Landolt ring L1, L2, L3 on the electronically controllable monitor unit is arranged outside each individual Landolt ring and can move around the respective Landolt ring in eight possible positions. Each of these positions is associated by spatial proximity with a possible gap position of the Landolt ring. With the aid of a push-button D, the person can move the mark M from one position to the next. A pressure on the push-button D tells the evaluation unit, to which the push-button is connected, that the current position is to be recorded.

1-17. (canceled)

18. A method for ascertaining and mapping data representing the optical imaging properties of an eye of a person, in which n different test images are displayed individually in succession on an electronically controllable monitor, are viewed by the person from a settable viewing angle and a settable distance and are identified by an input comprising:
a) ascertaining a characteristic viewing angle value at a given distance at which a first probability for correct identification of the test images is achieved;
b) ascertaining another characteristic distance value while maintaining the viewing angle value, at which a second probability for correct identification is achieved which is greater than the first probability;
c) repeating steps a) and b), wherein in each case in step a) the another characteristic distance value ascertained in step b) is used as the given distance until the distance at which the person views the test image corresponds to a final distance; and
d) displaying ascertained characteristic viewing angle values and characteristic distance values in accordance with viewing angle and distance.

19. The method according to claim 18, wherein step a) is performed by the following sub-steps:
a′) selecting a first test image from the n test images and displaying the selected test image by an electronically controllable monitor so that the first test image is displayed at a distance from the eye of the person at a first given distance and is viewed from a first viewing angle;
b′) activating an input, by the person, to generate data from test information identifying the test image;
c′) classifying the test information as correct or incorrect depending on whether the person has correctly or incorrectly recognized the viewed test image;
d′) selecting a further test image from the n test images and displaying the further selected test image by the electronically controllable monitor so that the test image is displayed at a distance from the eye of the person at the previously set distance and is viewed from a further viewing angle that is smaller than the previous viewing angle, provided that the prior classified test information is correct, and repeating steps b′) to d′) until a classified test information is incorrect;
e′) selecting a further test image from the n test images and displaying the selected test image with the electronically controllable monitor so that the test image is displayed at a distance from the eye of the person at the previously set distance and is viewed from a further viewing angle that is larger than the previous viewing angle, and repeating steps b′), c′) and e′) until data from test information is correct; and
f′) repeating steps d′) and e′) until a convergence criterion for the viewing angle is met, in which the viewing angle remains within a predefinable tolerance range in spite of repeated execution of steps d′) and e′) in order to obtain the viewing angle value characteristic of the set distance.

20. The method according to claim 19, wherein:
selecting a reduction measure by which the viewing angle is reduced in step d′ to be greater than an enlargement measure by which the viewing angle is enlarged in step e′.

21. The method according to claim 19, wherein step b) is performed by the following sub-steps:
g′) selecting a further test image from the n test images and displaying the selected test image by the electronically controllable monitor so that the test image is displayed at a distance from the eye of the person at a distance greater than the previous distance and is viewed from an unchanged viewing angle provided that data from the test information is incorrect, and repeating b′), c′) and g′);
h′) selecting a further test image from the n test images and displaying the selected test image by the electronically controllable monitor so that the test image is displayed at a distance from the eye of a person at a distance shorter than the previous distance and is viewed from an unchanged viewing angle provided that data from the test information is correct, and repeating steps b′), c′) and h′); and
i′) repeating steps g′) and h′) until a convergence criterion for the distance from the person is met, with which the distance remains within a predefinable tolerance range in spite of repeated execution of steps g′) and h′) in order to obtain the distance value characteristic of the set viewing angle.

22. The method according to claim 20, wherein step b) is performed by the following sub-steps:
g′) selecting a further test image from the n test images and displaying the selected test image by the electronically controllable monitor so that the test image is displayed at a distance from the eye of the person at a distance greater than the previous distance and is viewed from an unchanged viewing angle provided that data from the test information is incorrect, and repeating b′), c′) and g′);
h′) selecting a further test image from the n test images and displaying the selected test image by the electronically controllable monitor so that the test image is displayed at a distance from the eye of a person at a distance shorter than the previous distance and is viewed from an unchanged viewing angle provided that data from the test information is correct, and repeating steps b′), c′) and h′); and
i′) repeating steps g′) and h′) until a convergence criterion for the distance from the person is met, with which the distance remains within a predefinable tolerance range in spite of repeated execution of steps g′) and h′) in order to obtain the distance value characteristic of the set viewing angle.

23. The method according to claim 21, wherein:
an enlargement measure by which the distance from the person is enlarged in step g′ is selected to be greater than a reduction measure by which the distance in step h′ is reduced.

24. The method according to claim 22, wherein:
an enlargement measure by which the distance from the person is enlarged in step g′ is selected to be greater than a reduction measure by which the distance in step h′ is reduced.

25. The method according to claim 18, wherein:
as a first probability R1, a value is selected that is greater than a probability, which is 1/n and as a second probability R2, a value is selected than is greater than R1, but is smaller than 1.

26. The method according to claim 19, wherein:
as a first probability R1, a value is selected that is greater than a probability, which is 1/n and as a second probability R2, a value is selected than is greater than R1, but is smaller than 1.

27. The method according to claim 20, wherein:
as a first probability R1, a value is selected that is greater than a probability, which is 1/n and as a second probability R2, a value is selected than is greater than R1, but is smaller than 1.

28. The method according to claim 21, wherein:
as a first probability R1, a value is selected that is greater than a probability, which is 1/n, and as a second probability R2, a value is selected than is greater than R1, but is smaller than 1.

29. The method according to claim 23, wherein:
as a first probability R1, a value is selected that is greater than a probability, which is 1/n and as a second probability R2, a value is selected than is greater than R1, but is smaller than 1.

30. The method according to claim 25, wherein:
R1<(1+1/n)/2 and
R2>(1+1/n)/2.

31. The method according to claim 18, wherein:
step a) is performed by the following sub-steps:
a″) selecting a first test image from n test images and displaying the selected test image by the electronically controllable monitor so that the first test image is displayed at a distance from the eye of the person at a first distance and is viewed from a first viewing angle;
b″) the person activates an input, in order to generate data from test information identifying the test image;
c″) classifying the data from the test information as correct or incorrect depending on whether the person has correctly or incorrectly recognized the viewed test image;
d″) repeating steps a″) to c″) and ascertaining a relative frequency for correct recognition of the first test image;
e″) selecting a further test image from the n test images and displaying the selected test image by the electronically controllable monitor so that the test image is displayed at a distance from the eye of the person at the previously set distance and is viewed from a further viewing angle that is smaller than the previous viewing angle, and repeating steps b″) and c″);
f′) repeating step e″) and ascertaining a relative frequency for correct recognition of the further test image; and
g″) repeating steps e″) and f′) wherein, with each repetition of step e″) and f″), the further viewing angle from which the person views the test image is reduced compared with the viewing angle in the step immediately before until the relative frequency corresponds to a lower limit value at which the viewing angle corresponds to the characteristic viewing angle.

32. The method according to claim 31, wherein step b) according to claim 31 is performed by the following sub-steps:
h″) selecting a further test image from the n test images and displaying the selected test image by the electronically controllable monitor so that the test image is displayed at a distance from the eye of the person at a distance that is greater compared with the previous distance and is viewed from an unchanged viewing angle, and repeating steps b″) and c″);
j″) repeating step h″) and ascertaining a relative frequency for correct recognition of the further test image; and
k″) repeating steps h″) and j″) wherein, with each repetition of step h″) and j″), the distance at which the person views the test image is enlarged compared with the distance in the step immediately before until the relative frequency corresponds to an upper limit value at which the distance corresponds to the characteristic distance.

33. The method according to claim 32, wherein:
the data representing the optical imaging properties of the eye of the person is displayed in a form of the relative frequencies in accordance with viewing angle and distance at least for the relative frequency equals the lower limit value and the relative frequency equals the upper limit value.

34. The method according to claim 32, wherein for the lower limit value and the upper limit value:
0.1≦the lower limit value≦0.3 and 0.7≦the upper limit value 1.

35. The method according to claim 33 wherein for the lower limit value and the upper limit value:
0.1≦the lower limit value≦0.3 and 0.7≦the upper limit value 1.

36. The method according to claim 16, wherein:
when n=8, 8 Landolt rings are used as test images.

37. The method according to claim 16, wherein:
the n different test images are displayed individually in succession in a pseudo-random repeated sequence on the electronically controllable monitor.

38. A device for ascertaining and mapping data representing the optical imaging properties of an eye of a person, comprising a vision test unit which has a chin rest and also an electronically controllable monitor which can be moved along a linear unit bidirectionally relative to the chin rest and on which a plurality of n different test images can be displayed individually, comprising:
a control for selecting individual test images that can be displayed on the monitor based on a selection rule by which a viewing angle from which the person views a test image for scaling a size of the individual test images, and controlling the linear unit based on a further selection rule to set a distance between the monitor and chin rest, from which the accommodation requirement is produced;
an input from which the person generates data from test information identifying the test image;
a classifier for classifying the test information as “incorrect” or “correct” depending on whether the person has correctly or incorrectly recognized the viewed test image;
an evaluation unit which, based on at least the first selection rule, ascertains a characteristic viewing angle value at least at one given distance by a reduction of the viewing angle, at which a first probability for correct identification of the test images can be achieved and based on at least a second selection rule, ascertains a characteristic distance value while maintaining the at least one ascertained characteristic viewing angle value by enlargement of a distance, at which a second probability for correct identification can be achieved, which is greater than the first probability; and
a display unit on which all ascertained characteristic viewing angle and distance values are displayable according to the viewing angle and the distance.

39. A device for ascertaining and mapping data, representing the optical imaging properties of an eye of a person, comprising:
a vision test unit, which has a chin rest, an electronically controllable monitor disposed fixedly relative to the chin rest and on which a plurality of n different test images can be displayed individually in sequence, and imaging optics disposed between the chin rest and the monitor and a control which selects individual test images that can be displayed on the monitor, on the basis of a selection rule, by which a viewing angle from which the person views a test image, scales a size of the individual test images and controls imaging optics based on a further selection rule to set a specific accommodation requirement for the person's view on the monitor by variation of a focus position that can be assigned to the imaging optics;
an input by which the person generates data of test information identifying the test image;
a classifier for classifying the test information as “incorrect” or “correct” depending on whether the person has correctly or incorrectly recognized the viewed test image;
an evaluation unit which based on at least the first selection rule, ascertains a characteristic viewing angle value at least at one given distance by iterative reduction of the viewing angle, at which a first probability for correct identification of the test images can be achieved and based on at least the second selection rule, ascertains a characteristic distance value while maintaining the at least one ascertained characteristic viewing angle value by enlargement of the distance at which a second probability for correct identification can be achieved, which is greater than the first probability; and
a display on which ascertained characteristic viewing angles and distance values can be displayed in accordance with viewing angle and distance.

40. The device according to claim 39, wherein:
the control causes an accommodation requirement to be controlled by controlling introducing into the imaging optics either optical lenses having a positive optical refractive value reducing the accommodation requirement or optical lenses having a negative optical refractive value increasing the accommodation requirement.

41. The device according to claim 38, comprising:
a memory unit in which at least the characteristic viewing angle and distance values can be stored.

42. The device according to claim 39, comprising:
a memory unit in which at least the characteristic viewing angle and distance values can be stored.

43. The device according to claim 40, comprising:
a memory unit in which at least the characteristic viewing angle and distance values can be stored.
